(19) United States
(12) Patent Application Publication
(10) Pub. No.: US 2026/0335135 A1
(43) Pub. Date: Oct. 29, 2026

(54) AQUEOUS HUMOR OUTFLOW DEVICE INSERTION APPARATUS FOR INSERTING AQUEOUS HUMOR OUTFLOW DEVICE FOR GLAUCOMA TREATMENT INTO EYE

(71) Applicant: Seoul National University Hospital, Seoul (KR)

(72) Inventor: Tae-Woo Kim, Seongnam-si (KR)

(73) Assignee: Seoul National University Hospital, Seoul (KR)

(21) Appl. No.: 19/107,013

(22) PCT Filed: Jul. 14, 2023
(86) PCT No.: PCT/KR2023/010112

§ 371 Date: Feb. 26, 2025

(30) Foreign Application Priority Data
Sep. 13, 2022 (KR) 10-2022-0115118

(51) Int. Cl. A61F 9/007; A61B 17/3205

(52) U.S. Cl. CPC A61F 9/00781; A61B 17/32053

(57) ABSTRACT

Provided is an aqueous humor outflow device insertion apparatus which enables an aqueous humor outflow device (AHOD) to be accurately inserted into a desired location within the eye. The apparatus comprises: a cylindrical housing; an injection needle which accommodates an AHOD and protrudes or retracts in a longitudinal direction of the housing through a sliding hole formed on top of the housing, to move between first and second positions; and a support having a lower end fixed to the housing and an upper end inserted into the injection needle. When the injection needle protrudes through the sliding hole to be placed in the first position, the support supports the lower part of the AHOD. When the injection needle retracts through the sliding hole to move to the second position, the AHOD is exposed to the outside by the movement of the injection needle and the support by the support.

### CROSS-REFERENCE TO RELATED APPLICATIONS

This application is a national phase application of PCT International Application No. PCT/KR 2023/010112, filed on Jul. 14, 2023, which claims the benefit of Korean Patent Application No. 10-2022-0115118, filed on Sep. 13 , 2022, both applications of which are incorporated herein by reference in their entirety.

### TECHNICAL FIELD

The disclosure relates to an aqueous humor outflow device insertion apparatus for inserting an aqueous humor outflow device, which can drain aqueous humor from an anterior chamber to an extraconjunctival space or subconjunctival space in glaucoma treatment, into an eye.

### BACKGROUND

Aqueous humor refers to a transparent fluid that occupies a space (referred to as an ‘anterior chamber’) between the cornea and iris of an eye and a space (referred to as a ‘posterior chamber’ between the iris and crystalline lens of the eye, and has properties similar to lymph. The aqueous humor serves to maintain intraocular pressure.

Meanwhile, glaucoma is a progressive optic neuropathy that causes abnormalities in the function of an optic nerve and leads to visual field defects. The main cause of glaucoma is damage to the optic nerve due to increased intraocular pressure. The intraocular pressure refers to the pressure inside the eye, and mainly depends on the balance of aqueous humor circulation. The aqueous humor is produced little by little every day in a ciliary body behind the iris, and flows out of the eye through circulation as much as it is produced. If too much aqueous humor is produced or less aqueous humor flows out due to circulatory disturbances, the pressure inside the eye rises. Such increased intraocular pressure causes the glaucoma. If the increase in the intraocular pressure continues, permanent vision loss may occur. Further, in the case of a weak optic nerve, damage to the optic nerve may occur due to the pressure even within a normal range of intraocular pressure, resulting in the glaucoma.

Because the glaucoma begins with the damage to the optic nerve and it is difficult to restore already damaged sites, the goal of all treatments is to prevent further damage. Treatments for decreasing the intraocular pressure with drugs are usually performed first. However, if various drugs have no effects on controlling the intraocular pressure or it is impossible to use the drugs due to side effects, surgical treatments such as trabeculectomy and aqueous humor outflow device insertion are applied.

Recently, there have been various attempts to use a microtube as an aqueous humor outflow device for the ease and safety purposes of surgery. However, the microtube has a length of only a few millimeters and a diameter of only a few hundred micrometers (um), and it is thus technically very difficult to accurately insert the aqueous humor outflow device into a specific location inside the eye.

### Technical Problem

The disclosure is to provide an aqueous humor outflow device insertion apparatus for accurately inserting an aqueous humor outflow device into a desired location inside an eye.

Problems to be solved in the disclosure are not limited to the forementioned problems, and other unmentioned technical problems can be clearly understood from the following description by those skilled in the art.

### Technical Solution

According to an embodiment of the disclosure, an aqueous humor outflow device insertion apparatus for inserting an aqueous humor outflow device for glaucoma treatment into an eye includes: a cylindrical housing; an injection needle which accommodates the aqueous humor outflow device therein and protrudes or retracts in a longitudinal direction of the housing through a sliding hole formed on a top of the housing to move between a first position and a second position; and a support having a lower end fixed to the housing and an upper end inserted into the injection needle. When the injection needle protrudes through the sliding hole to be placed in the first position, the support supports a lower portion of the aqueous humor outflow device accommodated in the injection needle. When the injection needle retracts through the sliding hole to move from the first position to the second position, the aqueous humor outflow device is exposed to an outside by movement of the injection needle and support of the support.

A slot formed on a lateral side of the housing may extend along the longitudinal direction. A lever connected to the injection needle and protruding outward through the slot may move together with the injection needle along the slot.

An elastic unit disposed between an upper end of the housing and the lever inside the housing may provide an elastic force to move the injection needle from the first position to the second position. A locking portion may be formed in the slot to prevent movement of the level upon the injection needle being located in the first position.

A buffer disposed between a lower end of the housing and a lever inside the housing may prevent the injection needle from rapidly moving from the first position to the second position.

A fixing unit coupled to the lower end of the support may be coupled to a lower end of the housing.

A pedestal protruding from a lateral side of the housing may be seated on a surface of the eye and prevent the housing from shaking upon the insertion of the injection needle.

Specific matters of other embodiments are included in the detailed description and the accompanying drawings.

### Advantageous Effects

As described above, in the aqueous humor outflow device insertion apparatus according to the disclosure, when the injection needle protrudes from the housing and is located in the first position, the injection needle with the aqueous humor outflow device accommodated therein may be accurately inserted in a specific position while piercing the sclera of the eye. Next, when the injection needle retracts into the housing and moves to the second position while the support supports one end of the aqueous humor outflow device within the injection needle, the aqueous humor outflow device is naturally exposed to the outside and maintained in the specific position. Accordingly, by the aqueous humor outflow device insertion apparatus according to the disclosure, the aqueous humor outflow device made of the microtube is easily and accurately inserted in the specific position within the eye.

When the level connected to the injection needle is located in the locking portion of the housing while the injection needle is located in the first position, the injection needle can piece the sclera of the eye stably because its longitudinal movement is restricted.

When the lever is released from the locking portion, the elastic unit automatically moves the injection needle from the first position to the second position, and thus an operator does not need to apply separate force to move the injection needle. Further, the buffer located on the opposite side of the elastic unit prevents the injection needle from moving rapidly from the first position to the second position and induces smooth and gentle movement of the injection needle.

If an operator's hand holding the housing trembles while the injection needle moves from the first position to the second position, the aqueous humor outflow device may be damaged. To prevent the damage, the pedestal is formed on the lateral side of the housing. When the injection needle moves to the second position with the pedestal seated on the surface of the eye, the shaking of the housing and the damage of the aqueous humor outflow device due to the operator's tremors are effectively prevented.

### BRIEF DESCRIPTION OF THE DRAWINGS

FIG. 1 is a perspective view of an aqueous humor outflow device insertion apparatus according to a first embodiment of the disclosure.

FIG. 2 is an exploded perspective view of the aqueous humor outflow device insertion apparatus of FIG. 1.

FIG. 3 is a longitudinal section view of the aqueous humor outflow device insertion apparatus of FIG. 1.

FIG. 4 is a perspective view of the aqueous humor outflow device insertion apparatus according to the first embodiment of the disclosure with an injection needle located in the second position.

FIG. 5 is a section view of an eye in which an aqueous humor outflow device is inserted by the aqueous humor outflow device insertion apparatus of FIG. 1.

FIG. 6 is a longitudinal section view of an aqueous humor outflow device insertion apparatus according to a second embodiment of the disclosure.

### DETAILED DESCRIPTION

The merits and features of the disclosure, and methods of achieving them will become apparent with reference to the embodiments described below in detail and the accompanying drawings. However, the disclosure is not limited to the embodiments set forth herein, but may be implemented in various forms. The following embodiments are provided in order to fully describe the disclosure and enable those skilled in the art, to which the disclosure pertains, to understand the disclosure, the scope of which is defined in the appended claims. Like numerals refer to like elements throughout.

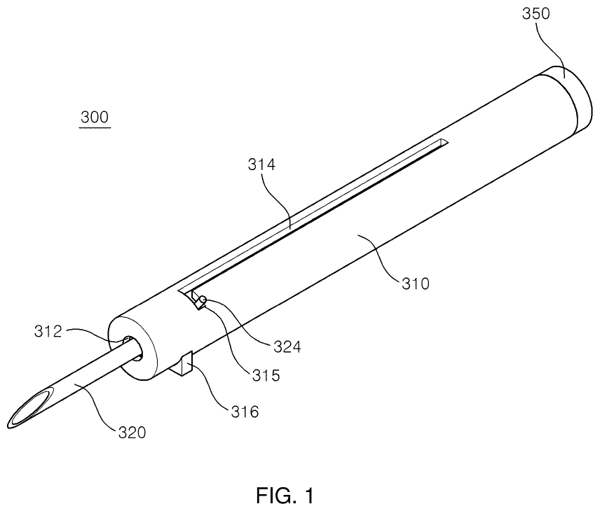

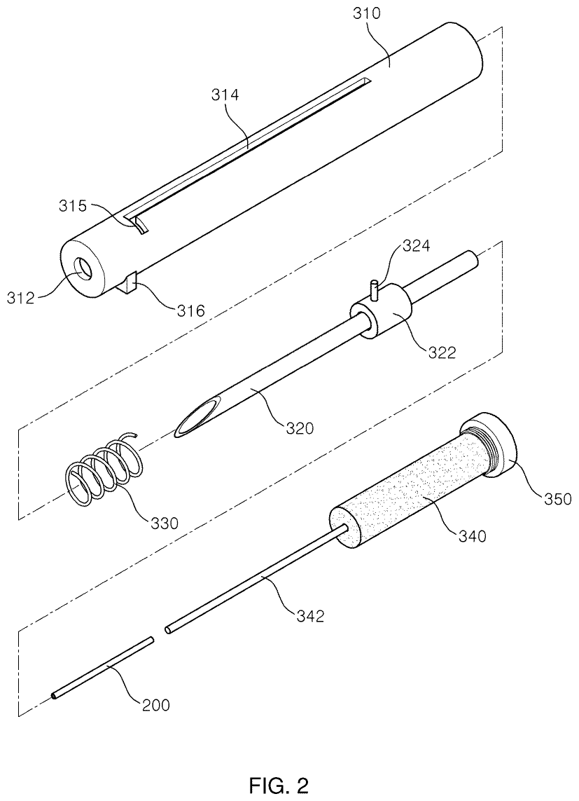

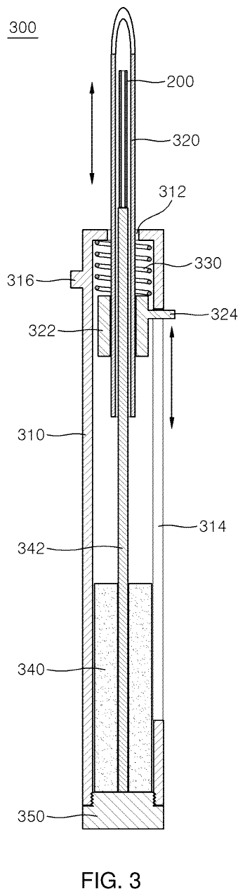

Below, an aqueous humor outflow device insertion apparatus according to a first embodiment of the disclosure will be described in detail with reference to FIGS. 1 to 3. FIG. 1 is a perspective view of an aqueous humor outflow device insertion apparatus according to a first embodiment of the disclosure. FIG. 2 is an exploded perspective view of the aqueous humor outflow device insertion apparatus of FIG. 1. FIG. 3 is a longitudinal section view of the aqueous humor outflow device insertion apparatus of FIG. 1.

The aqueous humor outflow device insertion apparatus 300 according to the first embodiment of the disclosure refers to an apparatus for inserting an aqueous humor outflow device 200 for glaucoma treatment into an eye 100, and includes a housing 310, an injection needle 320, a lever 324, a support 342, an elastic unit 330, a buffer 340, and a fixing unit 350. Here, the aqueous humor outflow device 200 has an overall tube shape and forms a passage through which aqueous humor 60 flows out from an anterior chamber 25 of the eye 100 to an extraconjunctival or subconjunctival space of the conjunctiva 30. The aqueous humor outflow device 200 may be made of a hollow fiber membrane, for example, polyether sulfone (PES), polytetrafluoroethylene (PTFE), or polyvinylidene difluoride (PVDF). As another example, the aqueous humor outflow device 200 may be made of silicone, collagen gel, poly(methyl methacrylate), or poly(styrene-block-isobutylene-block-styrene).

The housing 310 has an overall cylindrical shape and may be made of, for example, plastic. A sliding hole 312 is formed on the top of the housing 310. The sliding hole 312 guides the injection needle 320 to move in a longitudinal direction, and the inner diameter of the sliding hole 312 may be larger than or equal to the outer diameter of the injection needle 320. The fixing unit 350 is coupled to the lower end of the housing 310. For example, the fixing unit 350 may be screw-coupled to the lower end of the housing 310.

A slot 314 formed on a lateral side of the housing 310 extends along the longitudinal direction of the housing 310. The slot 314 may be formed as an opening extending on the lateral side of the housing 310 along the longitudinal direction.

The injection needle 320 accommodates the aqueous humor outflow device 200 therein, and is at least partially disposed inside the housing 310. The injection needle 320 protrudes or retracts through the sliding hole 312 in the longitudinal direction of the housing 310 and moves between the first position and the second position. For example, the injection needle 320 may protrude outward from the housing 310 through the sliding hole 312 and be located in the first position. The injection needle 320 may retract into the housing 310 through the sliding hole 312 and be located in the second position. The injection needle 320 may be made of a hard material, for example, stainless steel or polypropylene.

A sliding guide unit 322 formed on the outer circumferential surface of the injection needle 320 is disposed inside the housing 310 and guides the injection needle 320 to move in the longitudinal direction. For example, the sliding guide unit 322 may have an annular structure, and the outer diameter of the sliding guide unit 322 may be smaller than or equal to the inner diameter of the housing 310.

The lever 324 is coupled to the sliding guide unit 322. The lever 324 is connected to the injection needle 320 via the sliding guide unit 322. The lever 324 may protrude outward from the sliding guide unit 322 through the slot 314 of the housing 310. The lever 324 may be connected to the injection needle 320 and move together with the injection needle 320 along the slot 314 in the longitudinal direction. The injection needle 320, the sliding guide unit 322 and the lever 324 may be made of the same material as a single body.

The support 342 has an overall bar shape, and includes an upper end inserted in the injection needle 320 and a lower end fixed to the housing 310. The support 342 may be directly fixed to the housing 310, or may be coupled to the fixing unit 350 and indirectly fixed to the housing 310. The support 342 is disposed on the longitudinal axis of the housing 310 and inserted into the injection needle 320 as being fixed to the housing 310 regardless of the movement of the injection needle 320.

When the injection needle 320 protrudes through the sliding hole 312 and is located in the first position, the aqueous humor outflow device 200 is placed inside the injection needle 320, and the support 342 supports the lower portion of the aqueous humor outflow device 200 within the injection needle 320. When the injection needle 320 retracts through the sliding hole 312 and is located in the second position, the aqueous humor outflow device 200 is naturally exposed to the outside by the movement of the injection needle 320 and the support of the support 342.

The elastic unit 330 is disposed inside the housing 310, preferably between the upper end of the housing 310 and the lever 324, and more preferably between the upper end of the housing 310 and the sliding guide unit 322. The elastic unit 330 provides elastic force to the injection needle 320 so that the injection needle 320 can move from the first position to the second position. The elastic unit 330 may include, for example, a spring.

A locking portion 315 is formed in the slot 314 as a means for preventing the movement of the lever 324 in the longitudinal direction. For example, the locking portion 315 may be formed by extending the opening from the slot 314 in a direction other than the longitudinal direction. When the injection needle 320 is located in the first position, the lever 324 may rotate about the longitudinal direction and move from the slot 314 to the locking portion 315, thereby preventing the lever 324 and the injection needle 320 from moving by the elastic unit 330 in the longitudinal direction.

The buffer 340 is disposed inside the housing 310, preferably between the lower end of the housing 310 and the lever 324, and more preferably between the lower end of the housing 310 and the sliding guide unit 322. When the injection needle 320 moves from the first position to the second position by the elastic unit 330 in the longitudinal direction, the buffer 340 prevents rapid movement of the injection needle 320 and induces smooth and gentle movement. The buffer 340 may, for example, be made of a sponge annularly shaped to surround the support 342.

A pedestal 316 is structured to protrude from the lateral side of the housing 310. During a glaucoma treatment procedure, the pedestal 316 is seated on the surface of the eye to prevent the housing 310 from shaking due to an operator's hand tremors and thus prevent damage to the aqueous humor outflow device 200 when the injection needle 320 enters the housing 310 and moves to the second position.

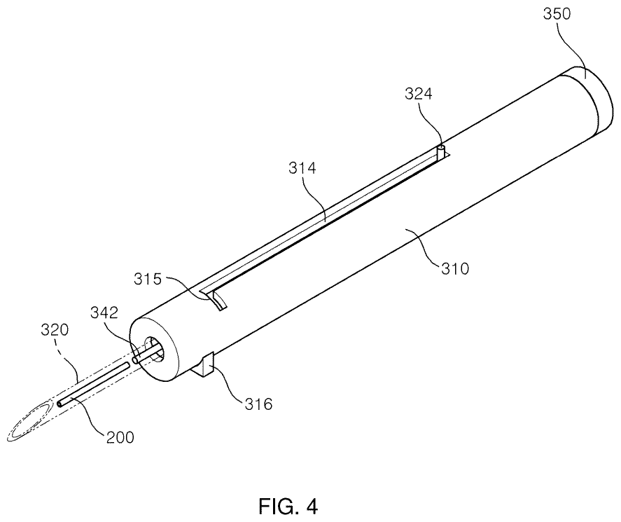

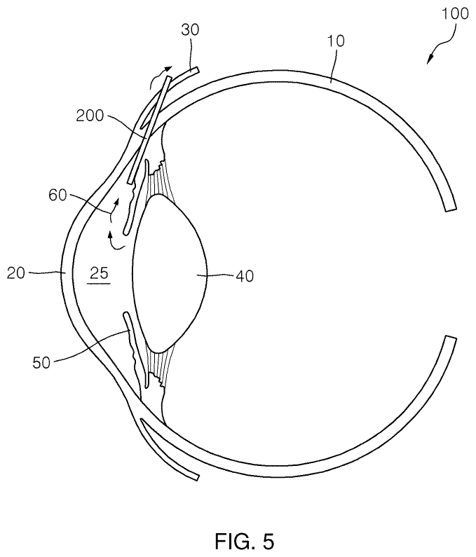

Below, the operations of the aqueous humor outflow device insertion apparatus according to the first embodiment of the disclosure will be described with reference to FIGS. 1, 4 and 5. FIG. 1 is a perspective view of the aqueous humor outflow device insertion apparatus according to the first embodiment of the disclosure with an injection needle located in the first position. FIG. 4 is a perspective view of the aqueous humor outflow device insertion apparatus according to the first embodiment of the disclosure with an injection needle located in the second position. FIG. 5 is a section view of an eye in which an aqueous humor outflow device is inserted by the aqueous humor outflow device insertion apparatus of FIG. 1.

Referring to FIGS. 1 and 5, when the injection needle 320 protrudes outward from the housing 310 through the sliding hole 312 and is located in the first position while accommodating the aqueous humor outflow device 200 therein, an operator inserts the injection needle 320 into a sclera 10 of the eye 100 and places it at a specific position. At this time, the movement of the lever 324 and the injection needle 320 may be prevented by the locking portion 315.

Next, referring to FIGS. 4 and 5, when an operator releases the lever 324 from the locking portion 315, the elastic unit 330 moves the injection needle 320 from the first position to the second position, and the lever 324 moves from a first end to a second end of the slot 314. In other words, while the injection needle 320 retracts into the housing 310 through the sliding hole 312, the aqueous humor outflow device 200 is naturally exposed to the outside and maintained at the specific position because it is supported by the support 342. In this embodiment, the injection needle 320 is entirely inserted into the housing 310 in the second position. However, the disclosure is not limited to this embodiment. In other words, the injection needle 320 may at least partially retract into the housing 310 so that the aqueous humor outflow device 200 can be exposed to the outside.

In FIG. 5, the reference numerals 20, 25, 30, 40, 50, and 60 not described above indicate the cornea, the anterior chamber, the conjunctiva, the crystalline lens, the iris, and the aqueous humor, respectively.

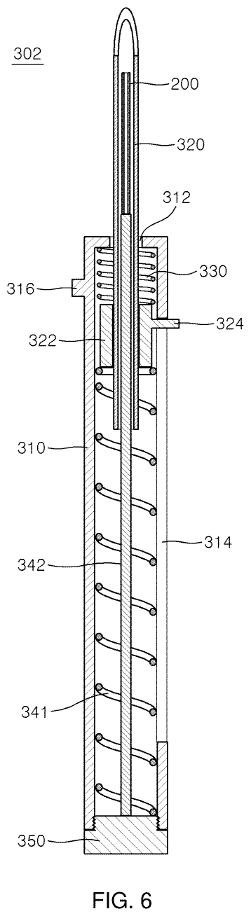

Below, an aqueous humor outflow device insertion apparatus according to a second embodiment of the disclosure will be described in detail with reference to FIG. 6. FIG. 6 is a longitudinal section view of an aqueous humor outflow device insertion apparatus according to a second embodiment of the disclosure. For the convenience of description, elements having the same function as those described in the first embodiment with reference to FIGS. 1 to 5 will be given the same reference numerals, and redundant descriptions thereof will be avoided. Below, descriptions will be made focusing on differences.

In an aqueous humor outflow device insertion apparatus 302 according to the second embodiment of the disclosure, the buffer 341 of is disposed inside the housing 310, preferably between the lower end of the housing 310 and the lever 324, and more preferably between the lower end of the housing 310 and the sliding guide unit 322. The buffer 341 prevents rapid movement of the injection needle 320 and induces smooth and gentle movement when the injection needle 320 moves from the first position to the second position by the elastic unit 330 in the longitudinal direction. The buffer 341 may include, for example, a spring surrounding the support 342.

Although a few embodiments of the disclosure have been described above with reference to the attached drawings, it will be understood by a person having ordinary knowledge in the art to which the disclosure pertains that the disclosure may be implemented in other specific forms without changing the technical ideas or essential features. Therefore, the embodiments described above should be construed in all respects as illustrative and not restrictive.

1. An aqueous humor outflow device insertion apparatus for inserting an aqueous humor outflow device for glaucoma treatment into an eye, the apparatus comprising:
a cylindrical housing;
an injection needle which accommodates the aqueous humor outflow device therein and protrudes or retracts in a longitudinal direction of the housing through a sliding hole formed on a top of the housing to move between a first position and a second position; and
a support having a lower end fixed to the housing and an upper end inserted into the injection needle, wherein
the support supports a lower portion of the aqueous humor outflow device accommodated in the injection needle, upon the injection needle protruding through the sliding hole to be placed in the first position, and
the aqueous humor outflow device is exposed to an outside by movement of the injection needle and support of the support, upon the injection needle retracting through the sliding hole to move from the first position to the second position.

2. The apparatus of claim 1, wherein
a slot formed on a lateral side of the housing extends along the longitudinal direction, and
a lever connected to the injection needle and protruding outward through the slot moves together with the injection needle along the slot.

3. The apparatus of claim 2, wherein
an elastic unit disposed between an upper end of the housing and the lever inside the housing provides an elastic force to move the injection needle from the first position to the second position, and
a locking portion is formed in the slot to prevent movement of the lever upon the injection needle being located in the first position.

4. The apparatus of claim 1, wherein a buffer disposed between a lower end of the housing and a lever inside the housing prevents the injection needle from rapidly moving from the first position to the second position.

5. The apparatus of claim 1, wherein a fixing unit coupled to the lower end of the support is coupled to a lower end of the housing.

6. The apparatus of claim 1, wherein a pedestal protruding from a lateral side of the housing is seated on a surface of the eye and prevents the housing from shaking upon the insertion of the injection needle.
